(19) United States
(12) Patent Application Publication
(10) Pub. No.: US 2026/0345134 A1
(43) Pub. Date: Nov. 5, 2026

(54) HAND-HELD DRILL WITH LONGITUDINAL MOVEMENT INDUCER

(71) Applicant: Distal Access, LLC, Park City, UT (US)

(72) Inventors: Emily Arbanas, Salt Lake City, UT (US); Kent Patrick Beck, Layton, UT (US); Shawn Patrick Fojtik, Park City, UT (US); Tim Nieman, North Salt Lake, UT (US)

(21) Appl. No.: 19/635,762

(22) Filed: Mar. 31, 2026

Related U.S. Application Data
(63) Continuation-in-part of application No. 19/633,998, filed on Mar. 30, 2026.
(60) Provisional application No. 63/781,347, filed on Mar. 31, 2025.
(60) Provisional application No. 63/780,443, filed on Mar. 30, 2025.

(51) Int. Cl. A61B 10/02; A61B 17/16; F16C 19/10; A61B 17/00; B23B 45/00

(52) U.S. Cl. CPC A61B 10/025; A61B 17/1626; A61B 17/1628; F16C 19/10; A61B 2017/00407; B23B 45/008; F16C 2316/10; F16C 2380/27

(57) ABSTRACT

A hand-operated and/or motor-driven drill includes a longitudinal movement inducer (e.g., a thrust bearing assembly, etc.) that enables for longitudinal movement of a tool rotated by the drill. The longitudinal movement inducer may facilitate introduction and/or advancement of the tool or a device driven by the tool into and/or through a substrate (e.g., a portion of a body of a subject, etc.). The longitudinal movement inducer may provide haptic feedback and/or audible feedback to a user. The drill may also be used to manually drive the tool. Methods for using the drill are also disclosed.

### CROSS-REFERENCE TO RELATED APPLICATIONS

A claim for priority to the Mar. 31, 2025, filing date of U.S. Provisional Patent Application No. 63/781,347, titled THRUST-BEARING ASSEMBLY, DRILLS INCLUDING THE THRUST-BEARING ASSEMBLY, AND METHODS OF USE (“the ’347 Provisional Application”), is hereby made pursuant to 35 U.S.C. § 119(e). This application is also a continuation-in-part of U.S. Patent Application No. 19/633,998, filed on Mar. 30, 2026, and titled ROOTER WITH SELECTIVE MANUAL AND MOTORIZED OPERATION (“the ’998 Application”), which claims priority to U.S. Provisional Patent Application No. 63/780,443, filed on Mar. 30, 2025, titled HAND-OPERATED AND/OR ELECTRIC DRILL AND METHODS OF USE (“the ’443 Provisional Application”), under 35 U.S.C. § 119(e). The entire disclosures of the ’443 Provisional Application, the ’347 Provisional Application, and the ’998 Application are hereby incorporated herein.

### TECHNICAL FIELD

This disclosure relates generally to drills and, more specifically, to motorized drills with spindles that rotate tools coupled to the spindles and with longitudinal movement inducers associated with the spindles to selectively induce longitudinal movement (e.g., repeated back-and-forth movement, or oscillating movement, such as vibration, hammering, etc.) into a rotated tool. This disclosure also relates to thrust bearing assemblies that, among other uses, can induce longitudinal movement in a spindle that drives a rotated tool. In addition, this disclosure relates to methods of selectively inducing longitudinal movement into a rotated tool while using the rotated tool.

### SUMMARY

In one aspect, a drill includes a housing, a motor, a spindle coupled to a shaft of the motor, a longitudinal movement inducer located along the shaft or in front of, or distal to, the shaft, and a coupler associated with the shaft or the longitudinal movement inducer. The drill also includes a handle defined by the housing and a trigger that actuates the motor.

The motor drives rotation of the shaft. As a sufficient reactive force is applied to the shaft (e.g., from pressure applied to a tool coupled to the coupler of the drill, etc.), the longitudinal movement inducer may be actuated, which may cause longitudinal movement (e.g., repeated back-and-forth movement, or oscillating movement, such as vibration, hammering, etc.) along the assembly of the shaft, the longitudinal movement inducer, the coupler, and any tool coupled to the coupler. More specifically, actuation of the longitudinal movement inducer may cause the coupler and any tool coupled to the coupler to repeatedly move back and forth. Such repeated back and forth movement may induce vibration and/or hammering in the coupler, any tool coupled to the coupler, the shaft, and the drill. Such vibration or hammering may facilitate introduction of the tool into or through a substrate by preventing the substrate from binding a tool or another device driven by the tool, by introducing additional force into the substrate, etc. Such vibration or hammering may also provide tactile feedback and/or audible feedback to a user of the drill, which may provide the user with information on the type of material into which the tool or a device driven by the tool is being introduced, other conditions encountered by the tool or the device driven by the tool, or the like.

The longitudinal movement inducer may be positioned on a distal portion of the spindle or distal to the spindle. The longitudinal movement inducer may induce longitudinal back-and-forth movement in the coupler of the drill, any tool coupled to the coupler (e.g., an elongated medical device (e.g., a sampling device, such as a biopsy needle, a needle, a cannula, a trocar, or a catheter; a wire; a macerator; a drill bit; etc.), a screwdriver, a drill bit, etc.), and any other device that may be driven by the tool (e.g., a screw, a bolt, etc.). Such longitudinal movement may be induced by way of concave features in and/or convex features on opposed surfaces of distal and proximal elements of the longitudinal movement inducer, which concave and/or convex features may repeatedly mate and uncouple to induce the longitudinal movement. The longitudinal movement inducer may enable the coupler, any tool coupled to the coupler, and any device driven by the tool to rotate without longitudinally moving the tool back and forth while the coupler, any tool coupled to the tool, any device driven by the tool, and a distal side of the longitudinal movement inducer rotate below a threshold reactive force and below a threshold resistance in rotation. The longitudinal movement inducer may induce the longitudinal back-and-forth movement in the tool when the coupler, any tool coupled to the coupler, any device driven by the tool, and/or the distal side of the longitudinal movement inducer encounter a reactive force that equals or exceeds the threshold reactive force and/or when the coupler, any tool coupled to the coupler, any device driven by the coupler, and/or the distal side of the longitudinal movement inducer encounter a resistance in rotation that equals or exceeds the threshold resistance in rotation. As another example, the longitudinal movement inducer may enable the coupler, any tool coupled to the coupler, and any device driven by the tool to rotate without longitudinally moving the coupler, the tool, and/or the device driven by the tool back and forth while the spindle and a proximal side of the longitudinal movement inducer rotate below a threshold rotational rate. The longitudinal back-and-forth movement may be induced as the rotational rate of the spindle and the proximal side of the longitudinal movement inducer reach or exceed the threshold rotational rate.

In various embodiments, the longitudinal movement inducer may comprise a thrust bearing. The thrust bearing may comprise a hub, a first thrust plate, a bearing cage carrying a plurality of bearings, and a second thrust plate.

The hub may include a proximal end and a distal end. The proximal end of the hub may couple to a distal end of the spindle of the drill or to a distal end of the coupler of the drill. The proximal end of the hub may enable the spindle to drive rotation of the hub while also enabling the hub to slide longitudinally back-and-forth over the distal end of the spindle or the coupler. The distal end of the hub may couple to a proximal end of a tool (e.g., an elongated medical device, a screwdriver, a drill bit, etc.) that is to be rotated as the hub rotates. A longitudinal position of the tool relative to the hub may be fixed as the proximal end of the tool is coupled to the distal end of the hub; thus, if the hub moves longitudinally relative to a spindle of a rooter, the tool will move longitudinally with the hub to substantially the same extent as the hub (e.g., accounting for a tool with a compressibility that exceeds a compressibility of the hub, etc.) or to the same extent as the hub.

The first thrust plate may also be referred to as a distal thrust plate. The first thrust plate may have a first outer surface (i.e., a distal side) and a first inner surface (i.e., a proximal side). The first inner surface may include one or more first interruptions (e.g., recesses (e.g., dimples, arcuate slots or grooves, radial grooves, etc.), protrusions (e.g., bumps, pimples, ridges, etc.), etc.); in embodiments where the first inner surface includes a plurality of first interruptions, which may comprise intermittent interruptions, the plurality of first interruptions may be arranged annularly about a center of rotation of the first thrust plate. The first thrust plate may reside over the hub. A position of the first thrust plate along the hub may be fixed. For example, the first thrust plate may be fixedly secured to the hub. As another example, the first thrust plate may be formed integrally with the hub.

The bearing cage may include a first side (i.e., a distal side) and a second side (i.e., a proximal side). The bearing cage may carry a plurality of bearings. The bearings may protrude from at least the first side of the bearing cage. Optionally, the bearings may also protrude from the second side of the bearing cage. The bearings may be arranged annularly around a center of rotation of the bearing cage. The bearing cage may reside over the hub, proximally adjacent to the first inner surface of the first thrust plate and with the first side of the bearing cage facing the first inner surface of the first thrust plate. The bearing cage may be able to slide longitudinally over the hub.

The second thrust plate may also be referred to as a proximal thrust plate. The second thrust plate may have a second inner surface (i.e., a distal side) and a second outer surface (i.e., a proximal side). Optionally, the second inner surface may include one or more second interruptions (e.g., recesses (e.g., dimples, arcuate slots or grooves, radial grooves, etc.), protrusions (e.g., bumps, pimples, ridges, etc.), etc.); in embodiments where the second inner surface includes a plurality of second interruptions, which may comprise intermittent interruptions, the plurality of second interruptions may be arranged annularly about a center of rotation of the second thrust plate. The second thrust plate may reside over the hub, proximally adjacent to the second side of the bearing cage and with the second inner surface of the second thrust plate facing the second side of the bearing cage. The second thrust plate may be able to slide longitudinally over the hub and may rotate (or not rotate) independently from rotation of the first thrust plate.

The thrust bearing may optionally include a spring. The spring may force the first thrust plate distally, away from one or both of the bearing cage, the bearing(s) carried by the bearing cage, and the second thrust plate.

A retainer may hold the thrust bearing in place relative to the distal end of the spindle in a manner that enables a proximal load to force the first thrust plate toward the second thrust plate. Such a retainer may include a distal limiter that may limit the distance the first thrust plate and the hub may move distally and, thus, may ensure that the thrust bearing remains assembled with the spindle of the drill. Such a retainer may also include a proximal limiter that may limit the distance the second thrust plate may move proximally toward a remainder of the drill as the first thrust plate may continue to move proximally toward the bearing cage and the second thrust plate. The retainer may be part of a housing for the thrust bearing or the drill.

The first interruption(s) and any optional second interruption(s) may be positioned to rotationally align with the bearings when the first thrust plate, the bearing cage, and the second thrust plate are assembled with the hub. Each of the first interruption(s) and optional second interruption(s) may interact with a bearing. When the bearing(s) interact with first interruption(s) and optional second interruption(s) that comprise recesses, the distance between the first thrust plate and the second thrust plate may be minimized and the first thrust plate. When the bearing(s) do not interact with first interruption(s) and optional second interruption(s) that comprise recesses, the first thrust plate and second thrust plate may be spaced a maximum contact distance apart from each other, which pushes the first thrust plate, the hub, and any tool (e.g., an elongated medical device, a screwdriver, a drill, etc.) coupled to the distal end of the hub distally. As the first thrust plate and second thrust plate move repeatedly between the minimum distance and maximum distance, the first thrust plate, the hub, and any tool coupled to the hub may move longitudinally (e.g., move repeatedly back and forth movement, or oscillate, such as vibrate, hammer, etc.).

When the bearing(s) interact with first interruption(s) and optional second interruption(s) that comprise protrusions, a contact distance between the first thrust plate and the second thrust plate may be maximized, which pushes the first thrust plate, the hub, and any tool (e.g., an elongated medical device, a screwdriver, a drill, etc.) coupled to the distal end of the hub distally. When the bearing(s) do not interact with first interruption(s) and optional second interruption(s) that comprise protrusions, the first thrust plate and second thrust plate may be spaced a minimum distance apart from each other. As the first thrust plate and second thrust plate move repeatedly between the minimum distance and maximum distance, the first thrust plate, the hub, and any tool coupled to the hub may vibrate or move in a hammering action.

Optionally, the drill may include a sprag clutch. The sprag clutch may be located at a distal end of the spindle or distal to the spindle. The sprag clutch may enable rotation of the coupler or tool coupled to the coupler to manually drive rotation of the coupler or tool (e.g., by rotating the drill about the coupler or tool, etc.) in a first rotational direction (e.g., clockwise, etc.) while enabling the drill to spin freely around the coupler or tool in a second rotational direction (e.g., counterclockwise, etc.). Such a sprag clutch may enable manual use of the drill; for example, in situations where the motor is unable to rotate the coupler or tool, in situations where slower rotation of the coupler or tool is desired, etc.

A method for introducing and/or advancing a tool or a device into a substrate may include coupling a tool to a coupler of a motorized drill, introducing the tool or a device driven by the tool into the substrate, and advancing the tool or a device driven by the tool at least partially through the substrate. Advancing the tool or a device driven by the tool may include powered operation of the drill, which may occur by pressing a trigger of the drill, and inducing longitudinal movement (e.g., repeated back-and-forth movement, or oscillating movement, such as vibration, hammering, etc.) in the tool while drilling. Longitudinal movement of the tool may be induced by applying a reactive force to the tool (e.g., under the weight of the drill, by applying pressure to the drill while drilling, etc.).

Longitudinal movement in the tool or a device driven by the tool may facilitate introduction of the tool or the device into or through a substrate by preventing the substrate from binding the tool or device, by introducing additional force into the substrate, etc. Longitudinal movement in the tool may also provide a user of the drill with tactile feedback and/or audible feedback, which may provide the user with information on the type of material into which the tool or a device driven by the tool is being introduced, other conditions encountered by the tool or the device driven by the tool, or the like.

In some embodiments, the tool or a device driven by the tool may be advanced manually. Manual advancement of the tool or a device driven by the tool may employ use of a sprag clutch and include rotating the drill in a first rotational direction (e.g., clockwise, etc.) to advance the tool or a device driven by the tool, then rotating the drill in a second rotational direction (e.g., counterclockwise, etc.) to again position the drill for rotation in the first rotational direction and, thus, further manual advancement of the tool or a device driven by the tool. Such repeated, back-and-forth rotational movement may be referred to as ratcheting.

In various embodiments, the drill may be used for medical purposes. Without limitation, the drill may be used to obtain a sample from a target location within a body of a subject. Such an embodiment of the method may include coupling a sampling device to a coupler of the drill, introducing a distal tip of the sampling device into the body of the subject, and advancing the distal tip of the sampling device through the body of the subject to the sample location. The sampling device may be advanced through the body of the subject by motorized operation of the drill, which may occur by pressing a trigger of the drill, and inducing longitudinal movement in the sampling device while drilling. Longitudinal movement of the sampling device may be induced by applying a reactive force to the sampling device (e.g., under the weight of the drill, by applying pressure to the drill while drilling, etc.). As the sampling device is advanced to the target location within the body of the subject, the sampling device may obtain the sample from the target location. The sampling device and the sample may then be removed from the body of the subject (e.g., by pulling the sampling device from the body, by reversing rotation of the sampling device with the drill, etc.). Of course, the drill may also be used for any of a variety of nonmedical purposes.

Other aspects of the disclosed subject matter, as well as features and advantages of various aspects of the disclosed subject matter should be apparent to those of ordinary skill in the art through consideration of the ensuing description, the accompanying drawings, and the appended claims.

### BRIEF DESCRIPTION OF THE DRAWINGS

In the drawings:

FIG. 1 is a side view of an embodiment of a drill of this disclosure, which includes a longitudinal movement inducer;

FIG. 2 is a cutaway side view showing interior features of various components of the embodiment of the drill shown in FIG. 1;

FIG. 3 is a partial assembly view showing embodiments of a motor, spindle, and longitudinal movement inducer of the embodiment of the drill shown in FIG. 1, as well as an embodiment of a tool that may be used with the drill;

FIG. 4 depicts the embodiments of the components shown in FIG. 3 in an assembled relationship;

FIGS. 5 and 6 show an embodiment of a gearing system that enables a user to select between manual use and motorized use of the drill;

FIG. 7 is a side view of an embodiment of a thrust bearing that may be used to induce longitudinal movement in a coupler of a drill, such as a coupler of the embodiment of the drill shown in FIG. 1, with the thrust bearing including a hub, a first thrust plate, a bearing cage, bearings, a second thrust plate, and a retainer;

FIG. 8 is an assembly view of embodiments of the first thrust plate, bearing cage, bearings, and second thrust plate shown in FIG. 7, with the first thrust plate and the second thrust plate oriented orthogonally, in different directions;

FIG. 9 is a partial cutaway view showing an embodiment of a manner in which the embodiment of the thrust bearing shown in FIG. 7 may be incorporated into the embodiment of the drill shown in FIG. 1;

FIG. 10 is a representation providing a side view of the embodiment of the thrust bearing shown in FIG. 7 without application of a load, or a proximally directed reactive force, to the coupler or a tool coupled to the coupler of the embodiment of the drill shown in FIG. 1;

FIG. 11 is a representation providing a side view of the embodiment of the thrust bearing shown in FIG. 7 with the application of a load, or a proximally directed reactive force, to the coupler or a tool coupled to the coupler of the embodiment of the drill shown in FIG. 1;

FIG. 12 is a cross-sectional representation of a portion of a thrust plate, showing an embodiment of a recess in the thrust plate;

FIG. 13 is a cross-sectional representation of a portion of a thrust plate, showing another embodiment of a recess in the thrust plate;

FIG. 14 is a perspective assembly view of other embodiments of a first thrust plate, bearing cage, bearings, and a second thrust plate;

FIG. 15 is an orthogonal view of an embodiment of a hub and a first thrust plate with an embodiment of protrusions on an inner surface of the first thrust plate;

FIG. 16 is an orthogonal view of another embodiment of a hub and a first thrust plate with another embodiment of protrusions on an inner surface of the first thrust plate; and

FIG. 17 depicts another embodiment of a drill that may include a longitudinal movement inducer.

### DETAILED DESCRIPTION

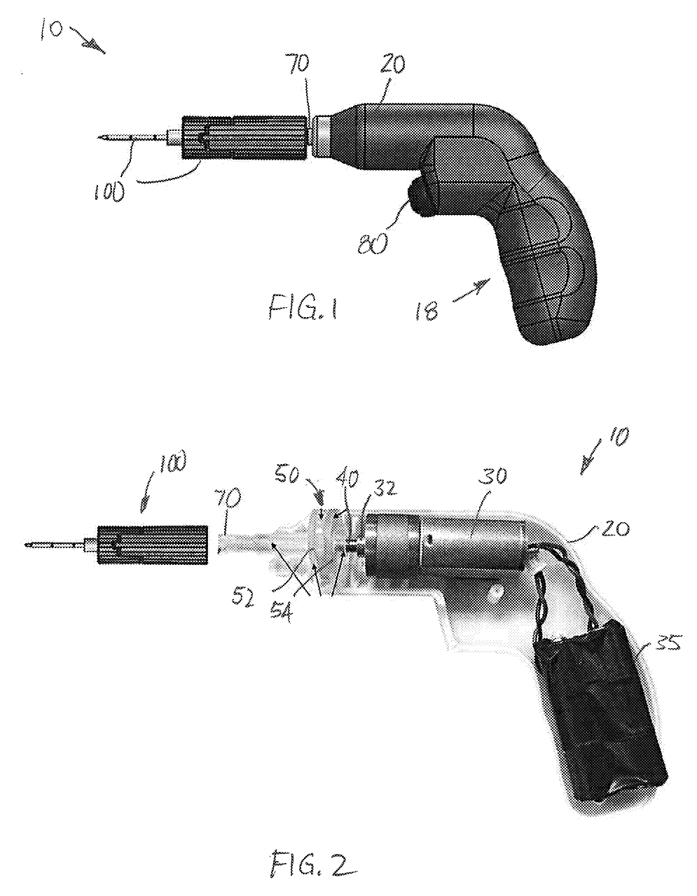

With reference to FIGS. 1-4, an embodiment of a drill 10 is depicted. The drill 10 may comprise a hand-held drill. The drill 10 may comprise a motorized drill. The drill 10 includes a housing 20, a motor 30, a spindle 40, a longitudinal movement inducer 50, a retainer 60, a coupler 70, and a trigger 80. The motor 30, spindle 40, longitudinal movement inducer 50, and trigger 80 may be carried by the housing 20. The retainer 60 may be carried by the housing 20 or defined by the housing 20. The spindle 40, longitudinal movement inducer 50, and coupler 70 may define a rotating assembly 75 of the drill 10, with the coupler 70 located at a distal end 77 of the rotating assembly 75.

The motor 30 may comprise any suitable electric motor (e.g., an electric motor with a shaft 32 that spins at up to 2000 rpm, etc.). A controller (not shown) may be associated with the motor 30. The controller may be programmed to automatically stop the motor 30 after a predetermined duration (e.g., 20 seconds, 30 seconds, etc.) of continuous use of the drill 10. The predetermined duration may be a maximum amount of time a user would normally use the drill 10 (e.g., in a medical procedure, etc.). Such a configuration may prevent overuse of the drill 10. Such a configuration may also enable the drill 10 to automatically shut down when it is accidentally and unknowingly turned on. In addition, such a configuration may save battery life and prevent accidental over heating of the drill 10.

The spindle 40 includes a proximal end 42 and a distal end 44. The proximal end 42 may be coupled to the shaft 32 of the motor 30. More specifically, the proximal end 42 may be coupled to the shaft 32 of the motor 30 in such a way that as the shaft 32 of motor 30 rotates the spindle 40 will rotate with the shaft 32.

The longitudinal movement inducer 50 may be coupled to or otherwise associated with the distal end 44 of the spindle 40. The longitudinal movement inducer 50 may slide longitudinally back-and-forth over the distal end 44 of the spindle 40.

The retainer 60 may hold the longitudinal movement inducer 50 in place relative to the distal end 44 of the spindle 40 in a manner that enables a proximally oriented load (e.g., the weight of the drill 10, a drilling force applied by a user to the drill 10, etc.) to force a distal side 54 of the longitudinal movement inducer 50 toward a proximal side 52 of the longitudinal movement inducer 50. Such a retainer 60 may include a distal limiter 64 that may limit the distance the distal side 54 of the longitudinal movement inducer 50 may move distally and, thus, may ensure that the longitudinal movement inducer 50 remains assembled with the spindle 40. Such a retainer 60 may also include a proximal limiter 62 that may limit the distance the distal side 52 of the longitudinal movement inducer 50 may move proximally toward the proximal end 42 of the spindle as the distal side 54 of the longitudinal movement inducer 50 may continue to move proximally toward the proximal side 52 of the longitudinal movement inducer 50. The retainer 60 may be part of a housing for the longitudinal movement inducer 50 or, as depicted, part of the housing 20 of the drill 10.

A configuration of the coupler 70 may enable it to engage a tool 100 that is to be used with and rotated by the drill 10. Without limitation, the coupler 70 may have a configuration that enables it to couple to an elongated medical device (e.g., a sampling device, such as a biopsy needle, a needle, a stylet needle, a stylet, a cannula, a stylet and cannula subassembly, a trocar, or a catheter; a wire; a macerator; a drill bit; etc.), a screwdriver, a drill bit, or the like.

The trigger 80 may be carried by a handle 18 of the drill 10. The handle 18 may be defined by the housing 20 of the drill 10. The trigger 80 may comprise a switch that, when depressed (e.g., with a user’s finger, etc.), supplies power to the motor 30 of the drill 10 to actuate the motor 30, or initiate operation of the motor 30, and, when released, terminates the supply of power to the motor 30 to terminate operation of the motor 30.

Optionally, the drill 10 may include a power supply 35. The power supply 35 may be carried by the housing 20 of the drill. The power supply 35 may supply power to the motor 30, with the trigger 80 controlling the supply of power to the motor 30. Without limitation, the power supply 35 may include one or more batteries. Alternatively, a remote power source (e.g., an external battery, mains power through an adaptor, etc.) may supply power to the motor 30 through the trigger 80.

Optionally, the drill 10 may include a sprag clutch 90. The sprag clutch 90 may be located at the distal end 44 of the spindle 40 or distal to the spindle 40. The sprag clutch 90 may enable rotation of the coupler 70 or tool 100 coupled to the coupler 70 to manually drive rotation of the coupler 70 or tool 100 (e.g., by rotating the drill 10 about the coupler 70 or tool 100, etc.) in a first rotational direction (e.g., clockwise, etc.) while enabling the drill 10 to spin freely around the coupler 70 or tool 100 in a second rotational direction (e.g., counterclockwise, etc.). Such a sprag clutch 90 may enable manual use of the drill 10; for example, in situations where the motor 30 is unable to rotate the coupler 70 or tool 100, in situations where slower rotation of the coupler 70 or tool 100 is desired, etc.

The sprag clutch 90 may comprise a bearing that allows for free rotation in one rotational direction (e.g., counterclockwise, etc.) without allowing for free rotation in an opposite rotational direction (e.g., clockwise, etc.). When included in a drill 10 of this disclosure, such a sprag clutch 90 may enable manual rotation of a tool 100 when desired or necessary (e.g., if the power supply 35 is drained (e.g., the battery runs out of power), power otherwise cannot be supplied to the motor 30, the motor 30 fails, etc.).

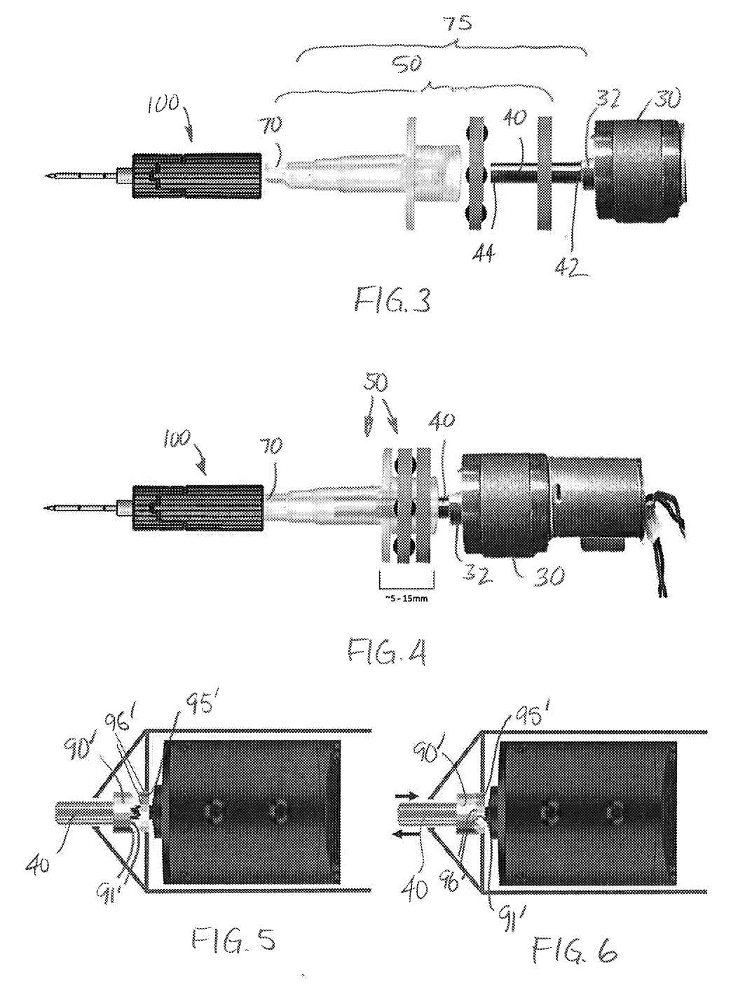

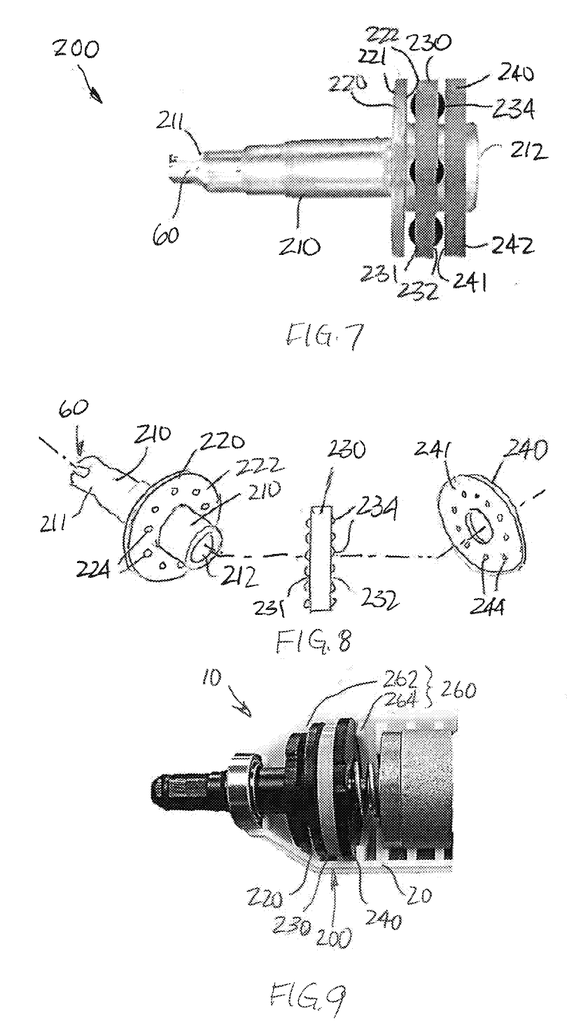

Alternatively, as shown in FIGS. 5 and 6, the drill 10 (FIGS. 1 and 2) may include a pair of gears 90′ and 95′ that function similarly to a sprag clutch 90 (FIGS. 1 and 2). For example, when under no compressive load, as shown in FIG. 5) the gears 90′ and 95′ may rotate freely relative to each other. As the gears 90′ and 95′ are compressed together, as shown in FIG. 6, they may disengage each other and rotate differently in one rotational direction but lock and rotate together in the opposite rotational direction. In a specific embodiment, one side of each tooth 91′, 96′ may be oriented at inclined angle (e.g., about 45°, etc.) while the other side of each tooth 91′, 96′ may be oriented substantially perpendicularly to allow cooperating teeth 91′ and 96′ to mate and uncouple when the motor 30 (FIGS. 1 and 2) of the drill 10 spins the spindle 40 (FIGS. 1 and 2) of the drill 10 counterclockwise but lock the teeth 91′ and 96′ while the motor 30 spins the spindle 40 clockwise. In such an embodiment, the motor 30 can spin a tool 100 (FIGS. 1 and 2) counterclockwise but, if the motor 30 fails, power is not available (e.g., a battery runs out, etc.), and/or the user wants to manually rotate the tool 100, the user can apply distal axial force to lock the gears 90 and 95 together, as shown in FIG. 6, and then rotate the entire drill 10 clockwise.

Turning now to FIGS. 7 and 8, an embodiment in which the longitudinal movement inducer 50 (FIGS. 1-4) of the drill comprises a thrust bearing 200 is depicted. As shown in FIGS. 7 and 8, the thrust bearing 200 may include a hub 210, a first thrust plate 220, a bearing cage 230, one or more bearings 234 carried by the bearing cage 230, and a second thrust plate 240.

The hub 210 may include a distal end 211 and a proximal end 212. The proximal end 212 of the hub 210 may couple to a distal end 44 (FIGS. 1-4) of the spindle 40 (FIGS. 1-4) of the drill 10 (FIGS. 1-4). The proximal end 212 of the hub 210 may enable the spindle 40 to drive rotation of the hub 210 while also enabling the hub 210 to slide longitudinally back-and-forth over the distal end 44 of the spindle 40. More specifically, the proximal end 212 of the hub 210 may include a receptacle 214 that receives the distal end 44 (FIGS. 1-4) of the spindle 40 (FIGS. 1-4). The receptacle 214 may have a shape (e.g., it may be keyed, etc.) that complements a shape of the distal end 44 of the spindle 40 to enable the hub 210 to rotate with the spindle 40. The distal end 211 of the hub 210 may couple to a proximal end of a tool 100 (FIGS. 1-4) (e.g., an elongated medical device, a screwdriver, a drill bit, etc.) that is to be rotated as the hub 210 rotates. A longitudinal position of the tool 100 relative to the hub 210 may be fixed as the proximal end of the tool 100 is coupled to the coupler 70, which may be defined by or coupled to the distal end 211 of the hub 210; thus, if the hub 210 moves longitudinally relative to a spindle 40 of a drill 10, the tool 100 will move longitudinally with the hub 210 to substantially the same extent as the hub 210 (e.g., accounting for a tool 100 with a compressibility that exceeds a compressibility of the hub 210, etc.) or to the same extent as the hub 210.

In a specific embodiment, the hub 210 may have a length of about 4 cm. The distal side of the hub 210 may have a length of about 3 cm. The proximal side of the hub 210 may have a length of about 1 cm. An outer diameter of the hub 210 may vary from about 5 mm or less (e.g., the outer diameter may be stepped down or taper distally, etc.) on a distal side of the first thrust plate 220 and about 1 cm on a proximal side of the first thrust plate 220.

The first thrust plate 220 may also be referred to as a distal thrust plate. The first thrust plate 220 may have a first outer surface 221 (i.e., a distal side) and a first inner surface 222 (i.e., a proximal side). The first inner surface 222 may include one or more first recesses 224 (e.g., dimples, arcuate slots, etc.); in embodiments where the first inner surface 222 includes a plurality of first recesses 224, the plurality of first recesses 224 may be arranged annularly about a center of rotation of the first thrust plate 220. Alternatively, the first inner surface 222 may include an annular groove that may receive the portions of the bearings 234 or the first inner surface 222 may be smooth. The first thrust plate 220 may reside over the hub 210. A position of the first thrust plate 220 along the hub 210 may be fixed. For example, the first thrust plate 220 may be fixedly secured to the hub 210. As another example, the first thrust plate 220 may be formed integrally with the hub 210.

The first thrust plate 220 may have an outer diameter of about 2.5 cm. A thickness of the first thrust plate 220 may be about 1 mm. Each recess 224 may have a depth of about 0.2 mm to about 0.5 mm.

The bearing cage 230 may include a first side 231 (i.e., a distal side) and a second side 232 (i.e., a proximal side). The bearing cage 230 may carry a plurality of bearings 234. The bearings 234, which may spin freely relative to the bearing cage 230, may protrude from at least the first side 231 of the bearing cage 230. Optionally, the bearings 234 may also protrude from the second side 232 of the bearing cage. The bearings 234 may be arranged annularly around a center of rotation of the bearing cage 230. The bearing cage 230 may reside over the hub 210, proximally adjacent to the first inner surface 222 of the first thrust plate 220 and with the first side 231 of the bearing cage 230 facing the first inner surface 222 of the first thrust plate 220. The bearing cage 230 may be able to slide longitudinally over the hub 210.

The bearing cage 230 may have an outer diameter of about 2.5 cm. An inner diameter of the bearing cage 230 may be about 1 cm. A thickness of the bearing cage 230 may be about 1 mm. Each bearing 234 may be spherical. A diameter of each bearing 234 may be about 1.25 mm to about 2 mm (e.g., 1.25 mm, 1.5 mm, 2 mm, etc.). By way of example and not limitation, smaller bearings 234 may be useful for drills 10 (FIGS. 1 and 2) and designed for intraosseous access into the proximal tibia and/or humorous with 15-gauge outer diameter (OD) needles, while larger bearings 234 may be useful for drills 10 designed for access into the iliac crest with larger 11-gauge OD needles.

The second thrust plate 240 may also be referred to as a proximal thrust plate. The second thrust plate 240 may have a second inner surface 241 (i.e., a distal side) and a second outer surface 242 (i.e., a proximal side). Optionally, the second inner surface 241 may include one or more second recesses 244 (e.g., dimples, arcuate slots, etc.); in embodiments where the second inner surface 241 includes a plurality of second recesses 244, the plurality of second recesses 244 may be arranged annularly about a center of rotation of the second thrust plate 240. Alternatively, in embodiments there the first inner surface 222 of the first thrust plate 220 includes one or more recesses 224, the second inner surface 241 may include an annular groove that may receive the portions of the bearings 234 or the second inner surface 241 may be smooth. The second thrust plate 240 may reside over the hub 210, proximally adjacent to the second side 232 of the bearing cage 230 and with the second inner surface of the second thrust plate 240 facing the second side 232 of the bearing cage 230. The second thrust plate 240 may be able to slide longitudinally over the hub 210. The second thrust plate 240 may rotate independently of the first thrust plate 220; in some embodiments, the second thrust plate 240 may remain rotationally stationary as the hub 210 and first thrust plate 220 rotate.

The second thrust plate 240 may have an outer diameter of about 2.5 cm. An inner diameter of the second thrust plate 240 may be about 1 cm. A thickness of the second thrust plate 240 may be about 1 mm. Each recess 244 may have a depth of about 0.2 mm to about 0.5 mm.

The depths of the recesses 224, 244 and the diameters of the bearings 234 may define the extent to which the first thrust plate 220 and second thrust plate 240 may move longitudinally while remaining in contact with the bearing cage 230.

The thrust bearing 200 may optionally include a spring 250. The spring 250 may be positioned at any location that may force the first thrust plate 220 distally, away from one or more of the bearing cage 230, the bearing(s) 234 carried by the bearing cage 230, and the second thrust plate 240. Of course, a thrust bearing 200 may function without a spring 250.

The first recess(es) 224 and any optional second recess(es) 244 may be positioned to rotationally align with the bearings 234 when the first thrust plate 220, the bearing cage 230, and the second thrust plate 240 are assembled with the hub 210. Each of the first recess(es) 224 and optional second recess(es) 244 may receive a bearing 234. When the bearing(s) 234 is (are) received within first recess(es) 224 and optional second recess(es) 244, the distance between the first thrust plate 220 and the second thrust plate 240 may be minimized and the first thrust plate 220, by way of the bearing(s) 234 and bearing cage 230, may intermittently engage the second thrust plate 240.

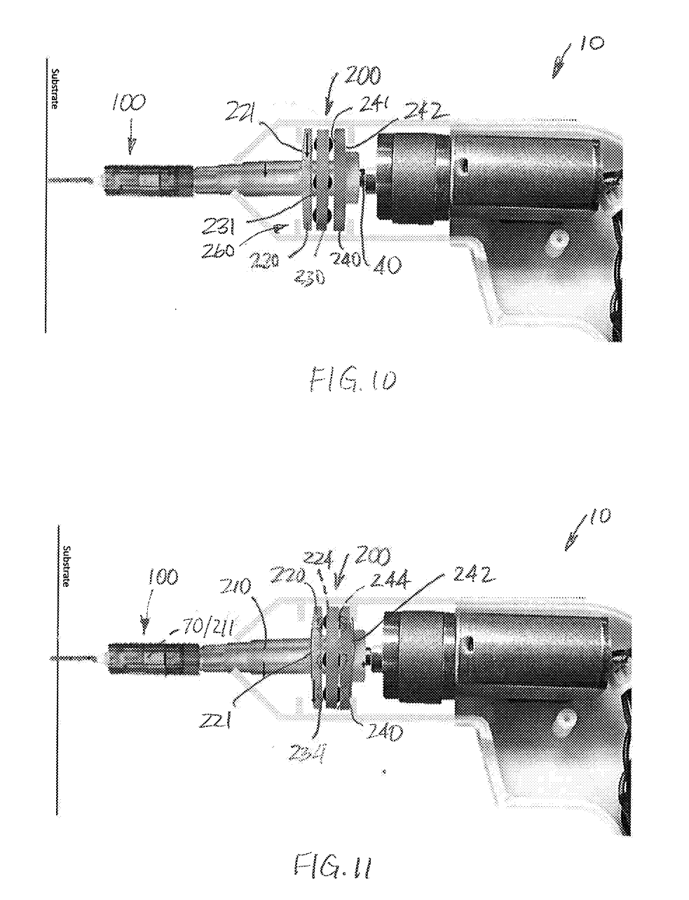

As shown in FIG. 9, the components of the thrust bearing 200 may be held together and in place relative to the spindle 40 of the drill 10 by way of a retainer 260. The retainer 260 may be carried by or defined by the housing 20 of the drill 10. The retainer 260 may hold the thrust bearing 200 in place relative to the distal end 44 of the spindle 40 in a manner that enables a proximal load to force (e.g., the weight of the drill 10, a drilling force applied by a user to the drill 10, etc.) the first thrust plate 220 toward the second thrust plate 240. Such a retainer 260 may include a distal limiter 262 that may limit the distance the first thrust plate 220 and the hub 210 may move distally and, thus, may ensure that the thrust bearing 200 remains assembled with the spindle 40 of the drill 10. Such a retainer 260 may also include a proximal limiter 264 that may limit the distance the second thrust plate 240 may move proximally toward a remainder of the drill 10 as the first thrust plate 220 may continue to move proximally toward the bearing cage 230 and the second thrust plate 240. In specific embodiments, the distal limiter 262 and proximal limiter 264 may be spaced about 6 mm to about 1 cm apart from each other.

As illustrated by FIG. 10, when no proximally oriented load is applied to the thrust bearing 200, the first thrust plate 220 may be located distally along the spindle 40 and distally within the retainer 260. At such a position, the first thrust plate 220 may be spaced apart from the bearings 234 protruding from the distal side 231 of the bearing cage 230. In a specific embodiment, a distance from the first outer surface 221 of the first thrust plate 220 to the second outer surface 242 of the second thrust plate 240 may be about 6 mm in such an arrangement. As the spindle 40 rotates, it may spin the tool 100 smoothly, without tactilely noticeable vibration. Thus, the drill 10 may function like an ordinary drill under little or no load.

As shown in FIG. 11, when a sufficient proximally oriented load is applied to the thrust bearing 200 (e.g., by way of a tool 100 that has been coupled to the coupler 70, etc.), the first thrust plate 220 may be pushed proximally. As the first thrust plate 220 is pushed proximally, the bearings 234 may intermittently engage the first recess(es) 224 of the first thrust plate 220 and/or any second recess(es) 244 of the second thrust plate 240, potentially placing the first thrust plate 220 and second thrust plate 240 a minimum contact distance apart from each other (e.g., less than 4 mm (e.g., about 3.8 mm, about 3.6 mm, about 3.5 mm, 3 mm, etc.) from the first outer surface 221 of the first thrust plate 220 to the second outer surface 242 of the second thrust plate 240 in the specific embodiment, etc.). When the sufficient proximally oriented load is applied to the thrust bearing 100 but the bearing(s) 234 is (are) not received within the first recess(es) 224 and the optional second recess(es) 244, the first thrust plate 220 and second thrust plate 240 may be spaced a maximum contact distance apart from each other (e.g., about 4 mm from the first outer surface 221 of the first thrust plate 220 to the second outer surface 242 of the second thrust plate 240 in the specific embodiment, etc.), which pushes the first thrust plate 220, the hub 210, and any tool 100 coupled to the distal end 211 of the hub 210 distally. As the first thrust plate 220 and second thrust plate 240 move repeatedly between the minimum distance and maximum contact distance, the first thrust plate 220, the hub 210, and any tool 100 coupled to the hub 210 move longitudinally (e.g., move repeatedly back and forth, or oscillate, such as vibrate, hammer, etc.) (e.g., the tool 100 may move back and forth by about 0.2 mm, about 0.25 mm, about 0.4, mm, about 0.5 mm, etc.). As the tool 100 moves longitudinally, the spindle 40 may continue to rotate the tool 100. The frequency with which the tool 100 moves longitudinally and/or the extent to which the tool 100 moves longitudinally may depend, at least in part on the amount of pressure a user applies to the drill 10 while using the drill 10.

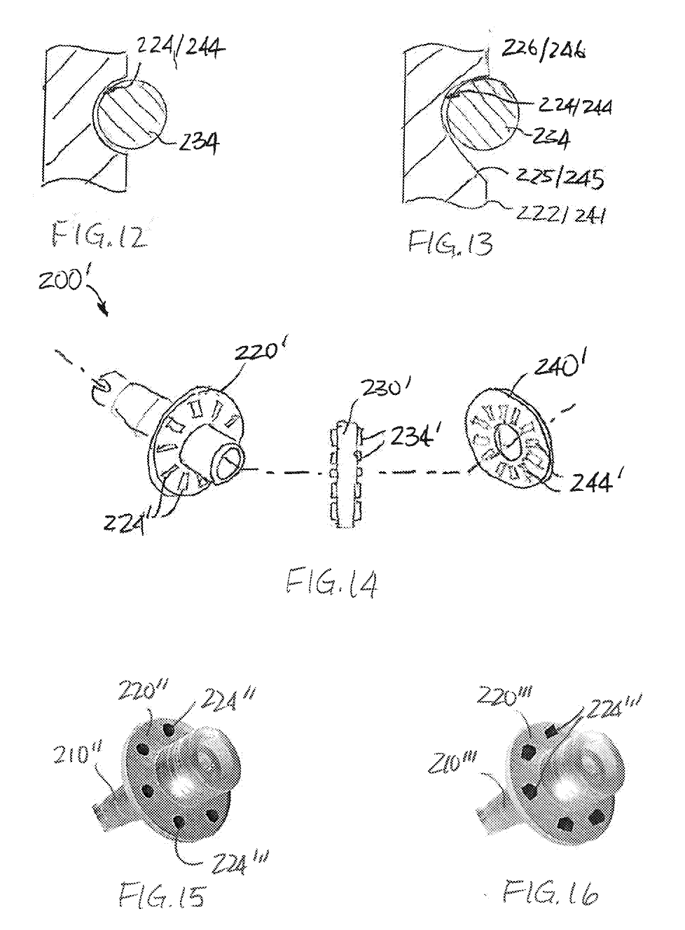

FIG. 12 shows an embodiment of a recess 224, 244, in which the recess 224, 244 comprises a dimple having the shape of part of a sphere (e.g., a hemispherical shape, a spherical dome shape, etc.). FIG. 13 shows an embodiment of a recess 224, 244, in which the recess has an entry side 225, 245 with a gradual slope and an exit side 226, 246 with a steeper slope or that is oriented substantially perpendicular to the surface 222, 241 in which the recess 224, 244 is formed. The entry side 225, 245 may enable a bearing 234 to smoothly enter the recess 224, 244, while the exit side 226, 246 may cause the bearing 234 to jump or bump as the bearing 234 exits the recess 224, 244.

In FIGS. 7, 8, 12 and 13, the bearings 234 are ball bearings, which are spherical in shape. FIG. 14 is a perspective assembly view of another embodiment of a thrust bearing 200′, which includes a bearing cage 230′ that carries one or more cylindrical bearings 234′, which are also referred to as roller bearings. The first thrust plate 220′ and second thrust plate 240′ include recesses 224′ and 244′, respectively, that are shaped to receive the cylindrical bearings 234′.

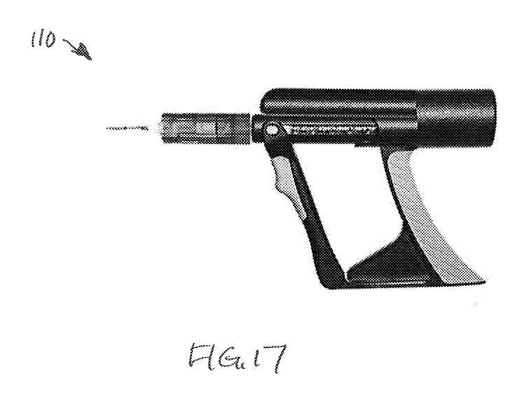

FIG. 15 is an orthogonal view of an embodiment of a hub 210′′ and a first thrust plate 220′′ with protrusions 224′′ on a first inner surface 222′′ of the first thrust plate 220′′. The protrusions 224′′ may be hemispherically shaped or spherical cap-shaped and may be referred to as pimples. Such a first thrust plate 220′′ may be part of a thrust bearing with a similarly configured second thrust plate or a differently configured second thrust plate, with or without a bearing cage. For example, the protrusions 224′′ may interact with bearings 234 (FIGS. 7 and 8) carried by a bearing cage 230 (FIGS. 7 and 8) or with interruptions (e.g., indentations, protrusions, etc.) on an opposite surface of a second thrust plate.

FIG. 16 is an orthogonal view of another embodiment of a hub 210′′′ and a first thrust plate 220′′′ with protrusions 224′′′ on a first inner surface 222′′′ of the first thrust plate 220′′′. The protrusions 224′′′ may comprise ridges or teeth. The ridges or teeth may be configured with an inclined angle on one side and a perpendicular angle on the other side to allow the teeth to mate then uncouple as a sufficient rotational resistance and/or reactive force is applied to the first thrust plate 220′′′. Such a first thrust plate 220′′′ may be part of a thrust bearing with a similarly configured second thrust plate or a differently configured second thrust plate, with or without a bearing cage. For example, the protrusions 224′′′ may interact with bearings 234 (FIGS. 7 and 8) carried by a bearing cage 230 (FIGS. 7 and 8) or with interruptions (e.g., indentations, protrusions, etc.) on an opposite surface of a second thrust plate. Without limitation, the first thrust plate 220′′′ and a complementary second thrust plate may be configured similarly to the ratcheting mechanisms disclosed by U.S. Patents 10,352,411, 11,002,346, and 12,390,203, the entire disclosures of which are hereby incorporated herein.

FIG. 17 illustrates an embodiment of a drill 110 that comprise a rooter of the type described by the ’998 Application. The drill 110, which may operate manually or under power of a motor, may also include a longitudinal movement inducer 50 (FIGS. 1-4) of this disclosure.

Referring again to FIGS. 1 and 2, in use, the drill 10 may rotate a tool 100 to facilitate introduction of the tool 100 or a device driven by the tool 100 into and, optionally, through a substrate. When the use is a medical use, the drill 10 may be used to introduce the tool 100 or the device driven by the tool 100 into the body of a subject.

As the drill 10 operates, it causes the tool 100 to rotate or spin in a drive direction. The drill 10 may also cause the rotated tool 100 to spin in a reverse direction. When sufficient pressure is applied to the drill 10 and the tool 100, a longitudinal movement inducer 50 of the drill 10 may induce longitudinal movement (e.g., repeated back-and-forth movement, or oscillating movement, such as vibration, hammering, etc.) in the tool 100 and in the drill 10. For example, pressing the drill 10 and the tool 100 or the device driven by the tool distally into a solid object may result in the application of a proximally directed reactive force to the tool 100 and, thus, to the coupler 70 and spindle 40 of the drill 10. The proximally directed reactive force may induce longitudinal movement in the tool 100 and in the drill 10. More specifically, longitudinal movement may be induced with a thrust bearing 200 (FIGS. 7-11), with a ratcheting mechanism, or the like. Such longitudinal movement of the tool 100 may be used to facilitate its introduction or the introduction of a device driven by the tool 100 into or through a hard structure (e.g., a blockage, such as arterial plaque; a calcification; bone; etc.).

As a user uses the drill 10, the drill 10 may vibrate in a manner indicative of a resistance of a substrate on the tool 100 or a device driven by the tool 100. For example, the harder the substrate, the greater the axial resistance to the tool 100 and the greater the extent to which the longitudinal movement inducer 50 will be compressed. This compression can result in the user feeling a corresponding amount of vibration, providing a haptic response to the substrate, and/or it may result in the user hearing the sound of such vibration. The harder the user pushes the drill 10 into the substrate and the harder the substrate, the more vibration and/or sound. With such haptic and/or audible feedback from the drill 10, a user can instantly increase, maintain, or decrease the amount pressure applied to the drill 10 based what they feel, hear, and see.

For example, when drilling through hard, dense cortical bone with a tool 100 on the drill 10, the hard, dense cortical bone may induce significant vibration in the drill 10. As the tool 100 moves through the hard, dense cortical bone into a softer cancellous and/or intramedullary space, the rotational resistance and, thus, the vibration and/or sound may decrease which may let the user know that they should modify the manner in which they are using the drill 10 and the tool 100. Sensing movement of the tool 100 into an area of less rotational resistance may prevent the user from over-driving the tool 100.

In some embodiments, the tool 100 or a device driven by the tool 100 may be advanced manually (e.g., if there is no power to the drill (e.g., if a battery of the drill 10 runs out of power, etc.), the motor 30 fails before or while drilling, the user wants to manually rotate or twist the tool 100, etc.). Manual advancement of the tool 100 or a device driven by the tool 100 may include employ use of an optional sprag clutch 90 of the drill 10 and include rotating the drill 10 in a first rotational direction (e.g., clockwise, etc.) under rotational resistance to advance the tool 100 or a device driven by the tool 100, then rotating the drill 10 freely in a second rotational direction (e.g., counterclockwise, etc.) to again position the drill 10 for rotation in the first rotational direction and, thus, further manual advancement of the tool 100 or a device driven by the tool 100. For example, a user may manually rotate the entire drill 10 clockwise from the 12:00 position to the 6:00 position causing the sprag clutch 90 to lock and manually drive the tool 100. Thereafter, the sprag clutch 90 may release as the user freely rotates the entire drill 10 counterclockwise from the 6:00 position back to the 12:00 position, enabling the user to again rotate the entire drill 10 clockwise. Such repeated, back-and-forth rotational movement may be referred to as ratcheting.

Although this disclosure provides many specifics, these should not be construed as limiting the scope of any of the claims that follow, but merely as providing illustrations of some embodiments of elements and features of the disclosed subject matter. Other embodiments of the disclosed subject matter, and of their elements and features, may be devised which do not depart from the spirit or scope of any of the claims. Features from different embodiments may be employed in combination. Accordingly, the scope of each claim is limited only by its plain language and the legal equivalents thereto.

What is claimed:

1. A drill, comprising:
a power-operated drill, including:
a body;
a spindle carried by the body and rotatable within the body;
a motor that selectively drives the spindle; and
a trigger that turns the motor on and off; and
a longitudinal movement inducer associated with the spindle.

2. The drill of claim 1, wherein the longitudinal movement inducer comprises a ratcheting mechanism along a length of the spindle.

3. The drill of claim 2, wherein the ratcheting mechanism includes:
a proximal member including a distal face and proximal teeth on the distal face; and
a distal member including a proximal face opposed to the distal face of the proximal member and distal teeth on the proximal face, the distal teeth configured to interact with the proximal teeth.

4. The drill of claim 3, wherein the proximal teeth and the distal teeth have a fully engaged arrangement that prevents longitudinal movement along or inline with the spindle and a partially disengaged arrangement that enables the longitudinal movement along or inline with the spindle.

5. The drill of claim 4, wherein one of the proximal member and the distal member rotate at different rates in the partially disengaged arrangement.

6. The drill of claim 1, wherein the longitudinal movement inducer comprises a thrust bearing.

7. The drill of claim 6, wherein the thrust bearing is positioned on a distal portion of the spindle.

8. The drill of claim 6, wherein the thrust bearing is positioned distal to the spindle.

9. The drill of claim 6, wherein the thrust bearing induces the longitudinal back-and-forth movement upon reaching a threshold resistance to rotation.

10. The drill of claim 6, wherein the thrust bearing induces the longitudinal back-and-forth movement upon reaching a threshold rotational rate.

11. A method for obtaining a sample from a target location within a body of a subject, comprising:
coupling a sampling device to a spindle of a drill;
introducing a distal tip of the sampling device into the body of the subject;
advancing the distal tip through the body of the subject to the sample location, including:
power driving the spindle of the medical drill by depressing the trigger of the medical drill to advance the sampling device toward the target location; and
moving the sampling device longitudinally back and forth to further advance the sampling device toward the target location; and
obtaining the sample at the target location with the sampling device.

12. The method of claim 11, wherein moving the sampling device longitudinally back and forth comprises inducing longitudinal back and forth movement of the sampling device with a thrust bearing.

13. The method of claim 12, wherein inducing longitudinal back and forth movement of the sampling device with the thrust bearing occurs as the thrust bearing encounters at least a threshold rotational resistance.

14. The method of claim 13, wherein inducing longitudinal back and forth movement of the sampling device with the thrust bearing occurs upon spinning the thrust bearing at a threshold rotational rate.

15. The method of claim 11, wherein moving the sampling device longitudinally back and forth comprises inducing longitudinal back and forth movement of the sampling device with a ratchet on the spindle.

16. A thrust bearing, comprising:
a first thrust plate comprising a first outer surface and a first inner surface;
an annular element carrying a plurality of rolling elements and including a first side and a second side, the rolling elements protruding from the first side and the second side, the first side facing the first inner surface of the first thrust plate; and
a second thrust plate comprising a second inner surface and a second outer surface, the second inner surface facing the second side of the annular element,
at least one of the first inner surface of the first thrust plate and the second inner surface of the second thrust plate including at least one recess that can receive a portion of a rolling element of the plurality of rolling elements.

17. The thrust bearing of claim 16, wherein each of the first inner surface of the first thrust plate and the second inner surface of the second thrust plate includes at least one recess.

18. The thrust bearing of claim 16, wherein the first inner surface of the first thrust plate and/or the second inner surface of the second thrust plate includes a plurality of recesses, a number of the plurality of recesses being the same as a number of the plurality of rolling elements.

19. The thrust bearing of claim 18, wherein each of the first inner surface of the first thrust plate and the second inner surface of the second thrust plate includes a plurality of recesses.

20. The thrust bearing of claim 16, wherein the first thrust plate rotates independently from the second thrust plate.
